(19) United States
(12) Patent Application Publication
(10) Pub. No.: US 2010/0105738 A1
(43) Pub. Date: Apr. 29, 2010

(54) EXTENDED RELEASE FORMULATIONS OF A PROTON PUMP INHIBITOR

(76) Inventors: Mitsuru Mizuno, Gifu (JP); Shigeru Aoki, Gifu (JP); Kiyoshi Iwamoto, Gifu (JP); Kenji Moroshima, Gifu (JP); Yasuhiro Zaima, Gifu (JP); Manabu Murakami, Tokyo (JP); Yukio Horai, Tokyo (JP); Takashi Seno, Ibaraki (JP); Norihiro Ueda, Ibaraki (JP)

Correspondence Address: NIXON & VANDERHYE, PC, 901 NORTH GLEBE ROAD, 11TH FLOOR, ARLINGTON, VA, 22203, US

(21) Appl. No.: 12/443,804

(22) PCT Filed: Oct. 4, 2007
(86) PCT No.: PCT/IB2007/004315

§ 371 Date: Mar. 31, 2009

Related U.S. Application Data
(60) Provisional application No. 60/850,023, filed on Oct. 6, 2006.

(51) Int. Cl. A61K 31/4439

(52) U.S. Cl. 514/338

(57) ABSTRACT

The invention provides extended release formulations of proton pump inhibitor, such as an extended release formulation of rabeprazole, comprising an amount of rabeprazole between 30 and 90 mg rabeprazole and having an AUC of between 900 and 1750 ng*hr/mL and other properties.

### RELATED APPLICATIONS

This application claims priority from U.S. Provisional Patent Application No. 60/850,023 filed on Oct. 6, 2006. The entire contents of this application are hereby incorporated by reference.

### FIELD OF INVENTION

The present invention relates to a controlled release or extended release pharmaceutical formulation of a proton pump inhibitor, wherein the formulation provides an AUC of a certain range; a Cmax of a certain range, or a combination thereof. An example of the present invention is a pulsed-release pharmaceutical composition containing a gastric acid secretion inhibitor.

### BACKGROUND

Rabeprazole sodium is a substituted benzimidazole proton pump inhibitor approved in some countries for treatment of duodenal ulcers, both erosive and nonerosive gastroesophageal reflux disease (GERD), GERD maintenance, Zollinger Ellison Syndrome (ZES), and for the eradication of Helicobacter pylori in combination with antibiotics.

The current immediate release formulation has an acid-suppression profile associated with acid breakthrough during the overnight period (nocturnal acid breakthrough, or NAB). NAB is defined as the occurrence of a nighttime gastric pH less than 4 for greater than one hour. NAB is believed to occur in more than 40% of patients treated with standard once or twice daily regimens of proton pump inhibitors. It would be desirable to provide improved gastric acid suppression, as measured by gastric pH.

### SUMMARY OF THE INVENTION

The invention provides extended release formulations of a proton pump inhibitor, such as rabeprazole, with certain pharmacokinetic properties and release properties that improve acid suppression, such as reducing nocturnal acid breakthrough (NAB).

One aspect of the invention provides an extended release pharmaceutical formulation of rabeprazole, comprising an amount of rabeprazole between 30 and 90 mg, wherein the AUC of the serum concentration profile of rabeprazole after administration of the formulation to a human subject is between 900 and 1750 ng·hr/mL.

Another aspect of the invention provides an extended release pharmaceutical formulation of rabeprazole, comprising between 30 and 90 mg of rabeprazole, wherein the Cmax of rabeprazole in the plasma serum of a human subject after administration of the formulation to the subject is between about 170 and about 440 ng/mL, preferably between 200.0 and 440.0 ng/mL.

Another aspect of the invention provides an extended release formulation of rabeprazole, comprising between 30 mg and 90 mg of rabeprazole, wherein Cmax of rabeprazole in the plasma serum of a human subject is between about 170 and about 440 ng/mL after administration of the formulation to the subject, preferably between 200.0 and 440.0 ng/mL and wherein AUC is between about 900 and about 1750 ng·hr/mL, preferably between 1000 and 1750 ng·hr/mL.

Another aspect of the invention is a method for providing an AUC of the serum concentration profile of rabeprazole after administration of the formulation to a human subject between about 900 and about 1750 ng·hr/mL, preferably between 1000 and 1750 ng·hr/mL, by administering to a patient a formulation of the invention. In one embodiment, a formulation of the invention is administered for a plurality of days, such as at least 2, at least 3, at least 4, at least 5, at least 6, or at least 7 days.

The extended release formulations are oral extended release formulations and the administration to the human subject is by oral administration.

Other aspects and embodiments of the invention are disclosed below.

### BRIEF DESCRIPTION OF THE FIGURES

FIG. 1 is a graph illustrating the results of dissolution tests for enteric tablet (A), pulsatile tablet (C), pulsatile tablet (D) and pulsatile tablet (F).

### DETAILED DESCRIPTION

Various aspects of the invention are described in the Summary section above. Described below are embodiments of the various aspects of the invention, such as a formulation with a certain amount of rabeprazole. These embodiments may also be combined, such as a certain amount of rabeprazole in a formulation with a certain Cmax or AUC range.

Embodiments of the various aspects of the invention include a formulation wherein the amount of rabeprazole present is (a) between 35 mg and 85 mg; (b) between 45 mg and 70 mg; (c) selected from 45 mg, 50 mg, 55 mg, 60 mg, 65 mg, 70 mg, and 80 mg; (d) selected from about 40 mg, about 50 mg, about 60 mg, and about 80 mg; (e) 40 mg; (f) 50 mg; (g) 60 mg; or (h) 65 mg.

Embodiments of the various aspects of the invention include a formulation wherein the rabeprazole is provided in the form of rabeprazole sodium, or another pharmaceutically acceptable rabeprazole salt.

Embodiments of the various aspects of the invention include formulations known in the art, such as oral dosage forms, for example, capsule dosage forms or tablet dosage forms or other formulations. The oral dosage form comprises an enteric formulation and a pulsed-release formulation of rabeprazole, for example, a capsule dosage form comprising both enteric tablets and pulsed-release tablets, e.g., a, pulsincap system dosage form.

In one embodiment, the enteric formulation technology (enteric tablet, enteric granule enteric capsule) provides substantially rapid release of rabeprazole in the intestine. One unexpected aspect of the invention is a pulsed release formulation that provides substantially zero order release of rabeprazole after desirable dissolution lag time of rabeprazole. Pulsed release from a pulsed release formulation (tablets or granules) is obtained by coating various coating components on the core comprising rabeprazole. For example, coating components of the coating layer are:

1) a mixture of water-insoluble polymer and enteric polymer and/or wax,

2) a mixture of water-insoluble polymer and water soluble polymer and/or wax, or

3) a mixture of wax and water soluble polymer.

Furthermore, in the case of 2) and 3), the enteric coating may be over-coated to stabilize the drug substance in the acidic fluid, such as gastric fluid.

The Pulsincap system dosage form (see, for example, WO90/09168; Wilding I R, Davis S S, Bakhshaee M, Stevens H N E, Sparrow R A, Brennan J. “Gastrointestinal transit and systemic absorption of captopril from a pulsed-release formulation.” Pharm Res. 1992; 9:654-657; and Saeger H, Virley P. Pulsincap & Mac226: Pulsed-Release Dosage Form. Product information from Scherer DDS, Ltd; 2004, the contents of each of which are hereby incorporated by reference in their entirety) is known as a pulsed release technique from capsule. The Pulsincap dosage form is composed of a water-insoluble capsule body enclosing a drug reservoir, and the body is closed at the open end with a swellable hydrogel plug. When this capsule comes in contact with the dissolution fluid, it swells; and after a desirable lag time, the plug pushes itself outside the capsule and rapidly releases the drug.

Embodiments of the various aspects of the invention include a formulation having a Cmax of rabeprazole in the plasma serum of a human subject after administration of the formulation to the subject (a) between about 180 and about 300 ng/mL; (b) between about 200 and about 440 ng/mL; (c) between about 200 and about 280 ng/mL; and (d) between about 200 and about 350 ng/mL. Preferred embodiments of the various aspects of the invention include a formulation having a Cmax of (a) between 180.0 and 300.0 ng/mL; (b) between 200.0 and 440.0 ng/mL; (c) between 200.0 and 280.0 ng/mL; and (d) between 200.0 and 350.0 ng/mL.

Other aspects of the invention include a method for increasing the percentage of time that intragastric pH is greater than (>) 4.0 during the 24-hour period after dosing, said method comprising administering to a patient a formulation of the invention. Embodiments of this invention include methods wherein the percentage of time is at least 70%, at least 75%, or at least 80%.

Furthermore, other aspects of the invention include a method for increasing the percentage of time that intragastric pH is greater than (>) 4.0 during the period of from about 14 hours to about 24 hours post-dose (for example, increasing intragastric pH during night-time, when the formulation is orally administered to human subject during the day), said method comprising administering to a patient a formulation of the invention. Embodiments of this invention include methods wherein the percentage of time is at least 50%, at least 60%, at least 65%, at least 70% or at least 75%.

It is believed that other proton pump inhibitors may be formulated in an analogous manner, according to the invention. Examples of proton pump inhibitors include omeprazole, esomeprazole, lansoprazole, pantoprazole and other benzimidazole derivatives with proton pump inhibitory action that suppresses gastric acid secretion.

### EXAMPLES

Additional information may be found in the publications WO 03/043661 (PCT/JP2002/12132), US Patent Pub. No. 2005/0163836, and WO/05/092336 (PCT/JP2005/005217), the contents of each of which are hereby incorporated by reference in their entirety.

### Example 1

TABLE 1
Components and Composition of Enteric Coated Tablet (A)

| | Amount |
|---|---|
| Component | mg |
| Core | |
| Rabeprazole sodium | 10 |
| Mannitol | 24.6 |
| Crospovidone | 16.5 |
| Sodium Hydroxide | 0.5 |
| Sodium Stearyl Fumarate | 0.9 |
| Dehydrated Ethanol ^{a} | q.s. |
| Core Weight (mg) | 52.5 |
| Under Coat | |
| Hydroxypropyl Cellulose | 2.17 |
| Calcium Stearate ^{b} | 0.73 |
| Dehydrated Ethanol ^{a} | q.s. |
| Under Coat Weight (mg) | 55.4 |
| Enteric Coat | |
| Hydroxypropylmethyl- | 6.64 |
| cellulose Phthalate | |
| Diacetylated | 0.66 |
| Monoglycerides | |
| Pigment Blend (yellow) ^{c} | 1.00 |
| Dehydrated Ethanol ^{a} | q.s. |
| Purified Water ^{a} | q.s. |
| Carnauba Wax | Trace |
| Total Weight (mg) | 63.7 |
| Ingredients | Content (w/w %) |
| Talc | 62.78 |
| Titanium dioxide | 33.33 |
| Ferric oxide (yellow) | 3.89 |

q.s. = quantum sufficit
^{a} Removed during drying
^{b} Vegetable origin is used.
^{c} Components and composition of pigment blend (yellow) are described below.

### Preparation of Enteric Tablet (A)

Uncoated tablets of the following composition were produced, an intermediate coating was coated thereon, and an enteric coating was subsequently applied.

An amount of 4.92 kg of mannitol and 3 kg of crospovidone were added to and mixed with 2 kg of rabeprazole sodium, 4 kg of ethanol having 0.1 kg of sodium hydroxide dissolved therein was added, and granulation was carried out. The granules thus produced were dried using a fluidized bed dryer, and then passed through a 1.5 mm screen. An amount of 0.3 kg of crospovidone and 0.18 kg of sodium stearyl fumarate were then added and mixed in, and tablet formation was carried out using a tablet machine. The uncoated tablets thus prepared each weighed 52.5 mg and contained 10 mg of rabeprazole sodium.

Next, an intermediate coating solution was obtained by dissolving 651 g of hydroxypropyl cellulose in 12.52 kg of ethanol and then uniformly dispersing 219 g of calcium stearate into the solution. The uncoated tablets were made to flow in a fluidized bed coating apparatus and the intermediate coating solution was sprayed on, thus forming an intermediate coating in an amount of 2.9 mg per tablet. The intermediate coating-covered tablets thus prepared each weighed 55.4 mg and contained 10 mg of rabeprazole sodium.

Moreover, separately, an enteric coating solution was prepared by (a) dissolving 1726 g of hydroxypropyl methyl cellulose phthalate and 172 g of glycerol fatty acid ester in 20.8 kg of 80% ethanol and (b) adding a suspension obtained by uniformly dispersing 260 g of pigment blend in 5.2 kg of an 80% ethanol solution. The enteric coating solution was sprayed onto the intermediate coating-covered tablets flowing in the fluidized bed coating apparatus, thus forming an 8.3 mg enteric coating. The enteric pharmaceutical composition thus produced contained 10 mg of rabeprazole sodium in a 63.7 mg tablet.

### Example 2

### Preparation of Tablets C, D, and F

TABLE 2
Components and Composition of Pulsatile Tablet (C)

| | Prescription |
|---|---|
| Component | Mg |
| Core | |
| Rabeprazole sodium | 10 |
| Mannitol | 24.6 |
| Crospovidone | 16.5 |
| Sodium Hydroxide | 0.5 |
| Sodium Stearyl Fumarate | 0.9 |
| Dehydrated Ethanol ^{a} | q.s. |
| Core Weight (mg) | 52.5 |
| Under Coat | |
| Hydroxypropyl Cellulose | 2.17 |
| Calcium Stearate ^{b} | 0.73 |
| Dehydrated Ethanol ^{a} | q.s. |
| Under Coat Weight (mg) | 55.4 |
| Pulsed Coat | |
| Ethylcellulose | 0.562 |
| Calcium Stearate | 2.376 |
| Methacrylic acid copolymer | 3.398 |
| Type A | |
| Talc | 0.594 |
| Titanium Dioxide | 0.357 |
| Tri-ethyl citrate | 0.713 |
| Dehydrated Ethanol ^{a} | q.s. |
| Carnauba Wax | Trace |
| Total Weight (mg) | 63.4 |

TABLE 3
Components and Composition of Pulsatile Tablet (D)

| | Prescription |
|---|---|
| Component | mg |
| Core | |
| Rabeprazole sodium | 10 |
| Mannitol | 24.6 |
| Crospovidone | 16.5 |
| Sodium Hydroxide | 0.5 |
| Sodium Stearyl Fumarate | 0.9 |
| Dehydrated Ethanol ^{a} | q.s. |
| Core Weight (mg) | 52.5 |
| Under Coat | |
| Hydroxypropyl Cellulose | 2.17 |
| Calcium Stearate ^{b} | 0.73 |
| Dehydrated Ethanol ^{a} | q.s. |
| Under Coat Weight (mg) | 55.4 |
| Pulsed Coat | |
| Ethylcellulose | 0.703 |
| Calcium Stearate | 2.970 |
| Methacrylic acid copolymer | 4.284 |
| Type A | |
| Talc | 0.743 |
| Titanium Dioxide | 0.445 |
| Tri-ethyl citrate | 0.891 |
| Dehydrated Ethanol ^{a} | q.s. |
| Carnauba Wax | Trace |
| Total Weight (mg) | 65.4 |

TABLE 4
Components and Composition of Pulsatile Tablet (F)

| | Prescription |
|---|---|
| Component | Mg |
| Core | |
| Rabeprazole sodium | 10 |
| Mannitol | 24.6 |
| Crospovidone | 16.5 |
| Sodium Hydroxide | 0.5 |
| Sodium Stearyl Fumarate | 0.9 |
| Dehydrated Ethanol ^{a} | q.s. |
| Core Weight (mg) | 52.5 |
| Under Coat | |
| Hydroxypropyl Cellulose | 2.17 |
| Calcium Stearate ^{b} | 0.73 |
| Dehydrated Ethanol ^{a} | q.s. |
| Under Coat Weight (mg) | 55.4 |
| Pulsed Coat | |
| Ethylcellulose | 0.984 |
| Calcium Stearate | 4.158 |
| Methacrylic acid copolymer | 5.946 |
| Type A | |
| Talc | 1.040 |
| Titanium Dioxide | 0.624 |
| Tri-ethyl citrate | 1.248 |
| Dehydrated Ethanol ^{a} | q.s. |
| Carnauba Wax | Trace |
| Total Weight (mg) | 69.4 |

Uncoated tablets of the following composition were produced, an intermediate coating was applied, and then an enteric coating was applied.

An amount of 4.92 kg of mannitol and 3 kg of crospovidone were added to and mixed with 2 kg of rabeprazole sodium, 4 kg of ethanol having 0.1 kg of sodium hydroxide dissolved therein was added, and granulation was carried out. The granules thus produced were dried using a fluidized bed dryer, and then passed through a 1.5 mm screen. An amount of 0.3 kg of crospovidone and 0.18 kg of sodium stearyl fumarate were then added and mixed in, and tablet formation was carried out using a tablet machine. The uncoated tablets thus prepared each weighed 52.5 mg and contained 10 mg of rabeprazole sodium.

Next, an intermediate coating solution was obtained by dissolving 651 g of hydroxypropyl cellulose in 12.52 kg of ethanol and then uniformly dispersing 219 g of calcium stearate into the solution. The uncoated tablets were made to flow in a fluidized bed coating apparatus and the intermediate coating solution was sprayed on, thus forming an intermediate coating in an amount of 2.9 mg per tablet. The intermediate coating-covered tablets thus prepared each weighed 55.4 mg and contained 10 mg of rabeprazole sodium.

Moreover, separately, an ethanol solution was obtained by (a) dissolving 1274.4 g of Eudragit L100 (methylacrylic acid copolymer Type A), 210.9 of ethyl cellulose and 267.3 g of triethyl citrate in 26.4 kg of ethanol, and (b) adding 891 g of calcium stearate, 222.9 kg of talc and 133.5 g of titanium dioxide and uniformly dispersing therein. The solution was sprayed onto the intermediate coating-covered tablets flowing in the fluidized bed so as to form an 8 mg, 10 mg or 14 mg coating. The controlled-release pharmaceutical composition thus produced contained 10 mg of rabeprazole sodium in a 63.4 mg tablet, 65.4 mg tablet or 69.4 mg tablet, referred to herein as tablets (C), tablets (D) and tablets (F), respectively.

### Example 3

### Preparation of G4 Capsule Dosage Form

Two enteric tablets (A), two tablets (C), two tablets (D) and two tablets (F) were filled into HPMC capsule (size No. 1). The filled capsule was vacuum dried at 40° C. for 10 hours.

### Example 4

### Preparation of G5 Capsule Dosage Form

An enteric tablet (A) and four tablets (D) were filled into HPMC capsule (size No. 1). The filled capsule was vacuum dried at 40° C. for 10 hours.

### Example 5

### Preparation of G6 Capsule Dosage Form

An enteric tablet (A) and four tablets (F) were filled into HPMC capsule (size No. 1). The filled capsule was vacuum dried at 40° C. for 10 hours.

### Example 6

### Preparation of G8 Capsule Dosage Form

Two enteric tablets (A) and four tablets (F) were filled into HPMC capsule (size No. 1). The filled capsule was vacuum dried at 40° C. for 10 hours.

### Example 7

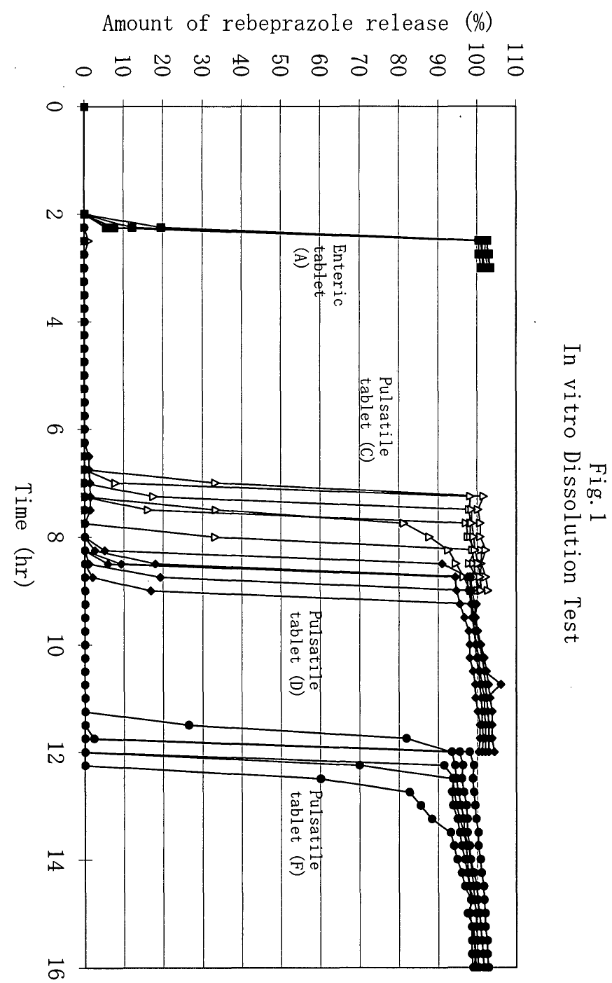

FIG. 1 shows the results of dissolution tests (n=6) for enteric tablet (A), pulsatile tablet (C), pulsatile tablet (D) and pulsatile tablet (F). The dissolution test was performed for 2 hours by the method described in the Japanese Pharmacopoeia (hereby incorporated by reference in its entirety) using an 0.1 N hydrochloric acid solution, followed by the dissolution test with the solvent replaced by 0.01 mol/L phosphate buffer (pH 6.8). The amount of rabeprazole released was measured using an ultra violet spectrophotometer (wavelength 290 nm).

The mean value of dissolution lag time of enteric tablet (A), pulsatile tablet (C), pulsatile tablet (D) and pulsatile tablet (F) was 2.0 hr, 7.29 hr, 8.50 hr and 11.83 hr, respectively. The dissolution lag time indicates the time taken for rabeprazole to start to dissolve in the test solution. Moreover, once the dissolution started to take place, the majority of rabeprazole in the formulation dissolved in a short time. At least 70% of rabeprazole dissolves within 3 hours, preferably within 2 hours, more preferably within 1 hours, after the desired dissolution lag time.

### Example 8

### Clinical Study

An open-label, parallel group, multi-dose study compared the intragastric pH profile of different rabeprazole extended release formulations, currently marketed 20 mg immediate release rabeprazole sodium and currently marketed 40 mg esomeprazole. H. pylori-negative healthy human volunteers were enrolled (about 31 volunteers per group) and orally administered once daily for 5 days with one of the formulations, selected from comparators and rabeprazole extended release formulations. Twenty-four hour intragastric pH was monitored on Days 1 and 5, and the plasma concentration of rabeprazole and its major metabolite PTBI (2-[[[4-(3-methoxypropoxy)-3-methyl-2-pyridinyl]-methyl]thio]-1H-benzimidazole) was measured after Day 5 dosing. One endpoint was the percentage of time the intragastric pH remained >4 during the 24-hour period after Day 5 dosing. Another endpoint was percentage of time the intragastric pH remained >4 during the period from 14 to 24-hours after dosing on Day 5 (see Table 5).

TABLE 5

| | | | | Total % of time | |
|---|---|---|---|---|---|
| | Total % of time | | P-value | intragastric pH > 4 | |
| | intragastric pH > 4 | Difference | compared | during the time from | Difference |
| | over a 24 hour | from | with | 14 hr to 24 hr after adminis- | from |
| | period on Day 5 Mean (SD) | Reference 1 | Reference 1 | tration on Day 5 Mean (SD) | Reference 1 |
| Reference 1 | | — | — | 32.8% (18.1%) | — |
| (Nexium | | | | | |
| Tablet 40 mg) | | | | | |
| Reference 2 | 63.2% (14.53) | −2.9% | — | 34.0% (23.09%) | −1.2% |
| (Enteric | | | | | |
| Tablet 20 mg) | | | | | |
| G5 | 78.9% (12.19) | 12.70% | P < 0.0001 | 62.5% (23.46) | 29.70% |
| G4 | 81.2% (11.42) | +15.1% | P < 0.0001 | 64.7% (21.36) | +31.9% |
| G6 | 84.1% (12.77) | +18.0% | P < 0.0001 | 72.4% (21.90) | +39.6% |
| G8 | 82.5% (15.36) | +16.4% | P < 0.0001 | 68.6% (26.97) | +35.8% |

As disclosed in Table 5, although the size of the groups was relatively small, nevertheless the extended release formulations demonstrated statistically significant and more than 10% improvement in the percentage of time that the intragastric pH remained >4 during the 24-hour period after Day 5 dosing when compared with esomeprazole. Each extended release formulation provided an intragastric pH of >4 during at least 70% of the 24-hour period after Day 5 dosing. Many of the formulations also provided a greater than 10% improvement in the percentage of time that intragastric pH remained >4 during the period from 14 to 24 hours after dosing on Day 5, compared with esomeprazole.

Pharmacokinetic parameters of rabeprazole were also measured after Day 5 dosing (See Table 6). Pharmacokinetic parameters of PTBI were also measured after Day 5 dosing (See Table 7). Means are expressed as geometric means (AUC0-t, Cmax) or arithmetic means (Tmax, Tlag, Tlast, Clast). Tlag is the time of the first observed plasma concentration, Tmax is the time of the maximum observed plasma concentration, Tlast is the time of the last observed plasma concentration, Cmax is the maximum observed plasma concentration, Clast is the last observed plasma concentration. AUC 0-t is the area under the plasma concentration-time curve from time zero to the time of last quantifiable plasma concentration.

Rabeprazole extended release formulations in each group demonstrated extended release of rabeprazole. Most human subjects had measurable plasma concentrations of rabeprazole sodium and its metabolite PTBI at up to 24 hours after the Day 5 dosing.

TABLE 6

| | Cmax | AUC 0-t | | | | |
|---|---|---|---|---|---|---|
| | (ng/ml) | (ng · hr/ml) | Tmax (hrs) | Tlag (hrs) | Tlast (hrs) | Clast (ng/ml) |
| | Mean (CV), | Mean (CV), | Mean (SD), | Mean (SD), | Mean (SD), | Mean(SD), |
| | Median | Median | Median | Median | Median | Median |
| Reference | 234.3 (58.42), | 447.3 (49.59), | 4.4 (3.16), | 2.4 (2.94), | 8.7 (3.67), | 9.8 (5.48), |
| 2 (Enteric | 243.1 | 504.3 | 4.0 | 1.02 | 8.0 | 7.60 |
| Tablet 20 mg) | | | | | | |
| G5 | 233.1 (37.48), | 1010 (36.23), | 4.7 (2.27), | 0.8 (1.06), | 20.0 (4.82), | 10.2 (4.91), |
| | 249.3 | 1031 | 5.0 | 0.50 | 24.0 | 8.80 |
| G4 | 400.2 (59.73), | 1663 (64.34), | 3.9 (1.61), | 0.5 (0.73), | 20.9 (4.50), | 13.5 (9.39), |
| | 398.8 | 1613 | 4.0 | 0 | 24.0 | 9.03 |
| G6 | 232.7 (43.82), | 1155 (67.37), | 4.2 (3.15), | 0.8 (2.10), | 22.0 (3.25), | 18.7 (20.3), |
| | 222.2 | 1113 | 3.0 | 0 | 24.0 | 9.87 |
| G8 | 422.5 (48.90), | 1616 (50.80), | 3.7 (2.05), | 0.5 (0.85), | 20.4 (4.78), | 12.9 (9.34), |
| | 413.1 | 1705 | 3.0 | 0 | 24.0 | 8.34 |

TABLE 7
Pharmokinetic Parameters of PTBI

| | Cmax | AUC 0-t | | | | |
|---|---|---|---|---|---|---|
| | (ng/ml) | (ng · hr/ml) | Tmax (hrs) | Tlag (hrs) | Tlast (hrs) | Clast (ng/ml) |
| | Mean (CV), | Mean (CV), | Mean (SD), | Mean (SD), | Mean (SD), | Mean (SD), |
| | Median | Median | Median | Median | Median | Median |
| Reference | 100.4 (49.51), | 688.1 (60.04), | 4.8 (1.78), | 1.1 (1.67), | 19.9 (5.78), | 12.9 (15.85), |
| 2 (Enteric | 107.9 | 828.4 | 5.0 | 0.0 | 24.01 | 7.86 |
| Tablet 20 mg) | | | | | | |
| G5 | 449.6 (37.68), | 4509 (38.63), | 8.7 (2.24), | 0.0 (0.0), | 24.0 (0.04), | 84.8 (55.71), |
| | 473 | 5090 | 8.0 | 0.0 | 24.00 | 65.41 |
| G4 | 476.4 (42.10), | 5105 (43.86), | 8.2 (1.82), | 0.1 (0.37), | 23.6 (1.59), | 90.6 (58.92), |
| | 493.1 | 5167 | 8.0 | 0.0 | 24.01 | 83.90 |
| G6 | 438.6 (45.77), | 5015 (51.85), | 10.3 (3.39), | 0.0 (0.0), | 23.8 (1.44), | 145.1 (95.76), |
| | 407.2 | 4662 | 10.0 | 0.0 | 24.00 | 115.8 |
| G7 | 437.5 (32.04), | 4792 (36.05), | 8.6 (2.00), | 0.0 (0.0), | 24.0 (0.06), | 95.1 (63.04), |
| | 426.7 | 5450 | 8.0 | 0.0 | 24.01 | 81.87 |

### Example 9

TABLE 8
Components and Composition of a Pulsed Release Tablet

| | Prescription |
|---|---|
| Component | mg |
| Core | |
| Rabeprazole sodium | 10 |
| Mannitol | 24.6 |
| Crospovidone | 16.5 |
| Sodium Hydroxide | 0.5 |
| Sodium Stearyl Fumarate | 0.9 |
| Dehydrated Ethanol ^{a} | q.s. |
| Core Weight (mg) | 52.5 |
| Under Coat | |
| Hydroxypropyl Cellulose | 2.17 |
| Calcium Stearate ^{b} | 0.73 |
| Dehydrated Ethanol ^{a} | q.s. |
| Under Coat Weight (mg) | 55.4 |
| Pulsed Coat | |
| Ethylcellulose | 5.0 |
| Calcium Stearate | 3.4 |
| Hydroxypropylcellulose | 3.0 |
| Talc | 0.8 |
| Titanium Dioxide | 0.6 |
| Tri-ethyl citrate | 1.2 |
| Dehydrated Ethanol ^{a} | q.s. |
| Pulsed Coat Weight (mg) | 69.4 |
| Enteric Coat | |
| Hydroxypropylmethyl-cellulose | 6.64 |
| Phthalate | |
| Diacetylated Monoglycerides | 0.66 |
| Pigment Blend (yellow) ^{c} | 1.00 |
| Dehydrated Ethanol ^{a} | q.s. |
| Purified Water ^{a} | q.s. |
| Carnauba Wax | Trace |
| Total Weight (mg) | 77.7 |

Uncoated tablets of the following composition are produced, an intermediate coating is applied, pulsatile release coating is applied, and then enteric coating is applied.

An amount of 4.92 kg of mannitol and 3 kg of crospovidone are added to and mixed with 2 kg of rabeprazole sodium, 4 kg of ethanol having 0.1 kg of sodium hydroxide dissolved therein is added, and granulation is carried out. The granules thus produced are dried using a fluidized bed dryer, and then pass through a 1.5 mm screen. An amount of 0.3 kg of crospovidone and 0.18 kg of sodium stearyl fumarate are then added and mixed in, and tablet formation is carried out using a tablet machine. The uncoated tablets thus prepared each weigh 52.5 mg and contain 10 mg of rabeprazole sodium.

Next, an intermediate coating solution is obtained by dissolving 651 g of hydroxypropyl cellulose in 12.52 kg of ethanol and then uniformly dispersing 219 g of calcium stearate into the solution. The uncoated tablets are made to flow in a fluidized bed coating apparatus and the intermediate coating solution is sprayed on, thus forming an intermediate coating in an amount of 2.9 mg per tablet. The intermediate coating-covered tablets thus prepared each weigh 55.4 mg and contain 10 mg of rabeprazole sodium.

Moreover, separately, an ethanol solution is obtained by (a) dissolving 750 g of hydroxypropylcellulose, 1250 of ethyl cellulose and 375 g of triethyl citrate in 30 kg of ethanol, and (b) adding 850 g of calcium stearate, 200 g of talc and 150 g of titanium dioxide and uniformly dispersing therein. The solution is sprayed onto the intermediate coating-covered tablets flowing in the fluidized bed so as to form 14 mg coating. The controlled-release pharmaceutical composition thus produced contains 10 mg of rabeprazole sodium in a 69.4 mg tablet.

Moreover, separately, an enteric coating solution is prepared by (a) dissolving 1726 g of hydroxypropyl methyl cellulose phthalate and 172 g of glycerol fatty acid ester in 20.8 kg of 80% ethanol and (b) adding a suspension obtained by uniformly dispersing 260 g of pigment blend in 5.2 kg of an 80% ethanol solution. The enteric coating solution is sprayed onto the controlled-release tablets flowing in the fluidized bed coating apparatus, thus forming an 8.3 mg enteric coating. The enteric pharmaceutical composition thus produced contains 10 mg of rabeprazole sodium in a 77.7 mg tablet.

### Example 10

### Capsule Dosage Form

An enteric tablet (A) and four tablets of Example 9, or two enteric tablet (A) and four tablets of Example 9 are filled into HPMC capsule (size No. 1). The filled capsule is vacuum dried at 40° C. for 10 hours.

### Example 11

TABLE 9
Components and Composition of a Pulsed release Tablet

| | Prescription |
|---|---|
| Component | mg |
| Core | |
| Rabeprazole sodium | 10 |
| Mannitol | 24.6 |
| Crospovidone | 16.5 |
| Sodium Hydroxide | 0.5 |
| Sodium Stearyl Fumarate | 0.9 |
| Dehydrated Ethanol ^{a} | q.s. |
| Core Weight (mg) | 52.5 |
| Under Coat | |
| Hydroxypropyl Cellulose | 2.17 |
| Calcium Stearate ^{b} | 0.73 |
| Dehydrated Ethanol ^{a} | q.s. |
| Under Coat Weight (mg) | 55.4 |
| Pulsed Coat | |
| Calcium Stearate | 8.4 |
| Hydroxypropylcellulose | 2.5 |
| Talc | 0.8 |
| Titanium Dioxide | 0.6 |
| Tri-ethyl citrate | 1.2 |
| Dehydrated Ethanol ^{a} | q.s. |
| Pulsed Coat Weight (mg) | 69.4 |
| Enteric Coat | |
| Hydroxypropylmethyl-cellulose | 6.64 |
| Phthalate | |
| Diacetylated Monoglycerides | 0.66 |
| Pigment Blend (yellow) ^{c} | 1.00 |
| Dehydrated Ethanol ^{a} | q.s. |
| Purified Water ^{a} | q.s. |
| Carnauba Wax | Trace |
| Total Weight (mg) | 77.7 |

Uncoated tablets of the following composition are produced, an intermediate coating is applied, pulse coating is applied, and then enteric coating is applied.

An amount of 4.92 kg of mannitol and 3 kg of crospovidone are added to and mixed with 2 kg of rabeprazole sodium, 4 kg of ethanol having 0.1 kg of sodium hydroxide dissolved therein is added, and granulation is carried out. The granules thus produced are dried using a fluidized bed dryer, and then pass through a 1.5 mm screen. An amount of 0.3 kg of crospovidone and 0.18 kg of sodium stearyl fumarate are then added and mixed in, and tablet formation is carried out using a tablet machine The uncoated tablets thus prepared each weigh 52.5 mg and contain 10 mg of rabeprazole sodium.

Next, an intermediate coating solution is obtained by dissolving 651 g of hydroxypropyl cellulose in 12.52 kg of ethanol and then uniformly dispersing 219 g of calcium stearate into the solution. The uncoated tablets are made to flow in a fluidized bed coating apparatus and the intermediate coating solution is sprayed on, thus forming an intermediate coating in an amount of 2.9 mg per tablet. The intermediate coating-covered tablets thus prepared each weigh 55.4 mg and contain 10 mg of rabeprazole sodium.

Moreover, separately, an ethanol solution is obtained by (a) dissolving 781.3 g of hydroxypropylcellulose and 375 g of triethyl citrate in 30 kg of ethanol, and (b) adding 2625 g of calcium stearate, 250 g of talc and 187.5 g of titanium dioxide and uniformly dispersing therein. The suspension is sprayed onto the intermediate coating-covered tablets flowing in the fluidized bed so as to form 13.5 mg coating. The controlled-release pharmaceutical composition thus produced contains 10 mg of rabeprazole sodium in a 68.9 mg tablet.

Moreover, separately, an enteric coating solution is prepared by (a) dissolving 1726 g of hydroxypropyl methyl cellulose phthalate and 172 g of glycerol fatty acid ester in 20.8 kg of 80% ethanol and (b) adding a suspension obtained by uniformly dispersing 260 g of pigment blend in 5.2 kg of an 80% ethanol solution. The enteric coating solution is sprayed onto the controlled-release tablets flowing in the fluidized bed coating apparatus, thus forming an 8.3 mg enteric coating. The enteric pharmaceutical composition thus produced contains 10 mg of rabeprazole sodium in a 77.7 mg tablet.

### Example 12

### Capsule Dosage Form

An enteric tablet (A) and four tablets of Example 11, or two enteric tablet (A) and four tablets of Example 9 are filled into HPMC capsule (size No. 1). The filled capsule is vacuum dried at 40° C. for 10 hours.

### Example 13

TABLE 10
Components and composition of active granules

| | Amount |
|---|---|
| Component | mg |
| Core | |
| Rabeprazole Sodium ^{a} | 40.0 |
| Mannitol Spheres ^{b} | 132.8 |
| Ethylcellulose | 7.2 |
| Dehydrated Alcohol ^{c} | q.s. |
| Core Weight (mg) | 180 |
| Coat | |
| Ethylcellulose | 7.8 |
| Hydroxypropylcellulose | 48.8 |
| Magnesium Stearate ^{d} | 22.4 |
| Dehydrated Alcohol ^{c} | q.s. |
| Coat Weight (mg) | 79 |
| Total Weight (mg) | 259 |

TABLE 11
Components and composition of enteric coated capsule

| Component | |
|---|---|
| Capsule filled with active | Amount |
| granules | mg |
| Active layer | |
| Rabeprazole sodium | 10.0 |
| Ethylcellulose | 1.8 |
| Dehydrated Alcohol ^{c} | q.s. |
| Undercoating layer | |
| Ethylcellulose | 1.8 |
| Hydroxypropylcellulose | 12.2 |
| Magnesium Stearate ^{d} | 5.6 |
| Dehydrated Alcohol ^{c} | q.s. |
| Enteric coating layer | |
| Ethylcellulose | 1.8 |
| Hydroxypropylcellulose | 12.2 |
| Magnesium Stearate ^{d} | 5.6 |
| Dehydrated Alcohol ^{c} | q.s. |
| Purified Water | q.s. |
| Total Weight (mg) | 68.7 |

q.s. = quantum sufficit.
^{a} Adjusted for potency and LOD.
^{b} DMF 19954. Holder: Freund.
^{c} Removed during drying process.
^{d} Vegetable origin is used.

### Preparation of Enteric Coated Capsules

### (Active Granule)

The ethanol solution of 200 g of rabeprazole sodium and 36 g of ethylcellulose is coated onto 664 g of mannitol spheres to form the granules containing rabeprazole.

Separately, an under coating solution was prepared by dissolving 78 g of ethylcellulose and 488 g of hydroxypropylcellulose and 224 g of magnesium stearate in 7 kg ethanol. The coating layer is coated onto the granules, continuously. The coated granules are active granules.

### (Enteric Coated Capsule)

An amount of 259 mg of the active granules are filled into a water-insoluble capsule and closed with a swellable hydrogel plug consisted of insoluble but permeable and swellable polymers (e.g., polymethacrylates), erodible compressed polymers (e.g., hydroxypropylmethyl cellulose, polyvinyl alcohol, polyvinyl acetate, polyethylene oxide), congealed melted polymers (e.g., saturated polyglycolated glycerides, glyceryl monooleate), and enzymatically controlled erodible polymer (e.g., pectin).

Separately, 3 g of rabeprazole sodium and 0.54 g of ethylcellulose are dissolved in 50 g of ethanol to prepare the coating solution to form the active layer. The 300 capsules described above filled with 259 mg of the active granules per a capsule are coated by the coating solution, and 10 mg of rabeprazole sodium is coated on a capsule.

Next, under-coating solution is obtained by dissolving 61 g of hydroxypropyl cellulose and 9 g of ethylcellulose in 1.25 kg of ethanol and then uniformly dispersing 28 g of calcium stearate into the solution. The capsules coated with 10 mg of rabeprazole sodium are made to flow in a pan coating apparatus and the intermediate coating solution is sprayed on, thus forming an intermediate coating in an amount of 19.6 mg per capsule. The under-coating covered capsules are prepared.

Moreover, separately, an enteric coating solution is prepared by (a) dissolving 159 g of hydroxypropyl methyl cellulose phthalate and 16.2 g of glycerol fatty acid ester in 2.08 kg of 80% ethanol and (b) adding a suspension obtained by uniformly dispersing pigment blend in 0.52 kg of an 80% ethanol solution. The enteric coating solution is sprayed onto the under-coating covered capsules flowing in the pan coating apparatus, thus forming a 33.3 mg enteric coating.

The enteric pharmaceutical capsule thus produced contains 50 mg of rabeprazole sodium in a capsule dosage form.

Each of the patents and publications cited herein is incorporated by reference herein in their entirety.

While this invention has been described with respect to various specific examples and embodiments, it is to be understood that the invention is not limited thereby. It will be apparent to one skilled in the art that various modifications can be made to the invention without departing from the spirit or scope of the appended claims.

1. An extended release pharmaceutical formulation of rabeprazole, comprising an amount of rabeprazole between 30 and 90 mg, wherein the AUG of the serum concentration profile of rabeprazole after administration of the formulation to a human subject is between 900 and 1750 ng·hr/mL.

2. A formulation of claim 1, wherein said amount of rabeprazole is between 35 mg and 85 mg.

3. A formulation of claim 1, wherein said amount of rabeprazole is between 45 mg and 70 mg.

4. A formulation of claim 1, wherein said amount of rabeprazole is selected from the group consisting of 45 mg, 50 mg, 55 mg, 60 mg, 65 mg, 70 mg, and 80 mg.

5. A formulation of claim 1, wherein said formulation is a capsule formulation.

6. A formulation of claim 1, wherein said rabeprazole is present in the form of a sodium salt of rabeprazole.

7. An extended release pharmaceutical formulation of rabeprazole, comprising between 30 and 90 mg of rabeprazole, wherein the Cmax of rabeprazole in the plasma serum of a human subject after administration of the formulation to the subject is between 170.0 and 440.0 ng/mL.

8. A formulation of claim 7, wherein Cmax is between 180.0 and 300.0 ng/mL.

9. A formulation of claim 7, wherein Cmax is between 200.0 and 430.0 ng/mL.

10. A formulation of claim 7, wherein said amount of rabeprazole is between 35 and 85 mg.

11. A formulation of claim 7, wherein said amount of rabeprazole is between 45 mg and 70 mg.

12. A formulation of claim 7, wherein said amount of rabeprazole is selected from the group consisting of 45 mg, 50 mg, 55 mg, 60 mg, 65 mg, 70 mg, and 80 mg.

13. An extended release formulation of rabeprazole, comprising between 30 mg and 90 mg of rabeprazole, wherein Cmax of rabeprazole in the plasma serum of a human subject is between 170.0 and 440.0 ng/mL after administration of the formulation to the subject and AUC is between 900 and 1750 ng·hr/mL.

14. A formulation of claim 1, wherein the formulation is an oral extended release formulation and the administration to the human subject is by oral administration.
